(19) United States
(12) Patent Application Publication
(10) Pub. No.: US 2016/0235306 A1
(43) Pub. Date: Aug. 18, 2016

(54) THERMAL MONITORING AND CONTROL

(71) Applicant: KONINKLIJKE PHILIPS N.V., Eindhoven (NL)

(72) Inventors: Louis Nicolas ATALLAH, Eindhoven (NL); Edwin Gerardus Johannus Maria BONGERS, Thorn (NL); Mohammed MEFTAH, Tilburg (NL); Ruslan Akhmedovich SEPKHANOV, Eindhoven (NL)

(21) Appl. No.: 15/026,773

(22) PCT Filed: Oct. 3, 2014
(86) PCT No.: PCT/IB2014/065045

§ 371 Date: Apr. 1, 2016

Related U.S. Application Data
(60) Provisional application No. 61/886,266, filed on Oct. 3, 2013.

(51) Int. Cl. A61B 5/01; A61F 7/00; A61B 5/00

(52) U.S. Cl. CPC A61B 5/015; A61B 5/4836; A61F 7/0097; A61B 5/6892; A61B 5/72; A61B 2503/045; A61N 2005/0651

(57) ABSTRACT

Systems and methods for non-invasive thermal monitoring use multiple coupling sensors and temperature sensors in order to determine multiple temperatures of a patient. The sensors may be carried by support structure such as a wrap, blanket, mattress, and similar structures. Signals generated by the coupling sensors reflect coupling strength and/or reliability between sensors and the patient. Positional information/mapping of temperature information can be derived from the coupling sensors, image sensors, and/or the variation of the temperature profile itself over time. The measurements may be used to construct a thermal (full-body) profile of the patient and provide targeted thermal control (heating/cooling).

### BACKGROUND

1. Field

The present disclosure pertains to a system and method for non-invasive determination of one or more temperatures, and, in particular, determining multiple temperatures in neonates.

2. Description of the Related Art

Measuring temperatures is known to be medically relevant. Reducing heat loss is particularly important for preterm neonates. Specifically, the core body temperature and the peripheral temperature are important measures for diagnostic purposes, including, but not limited to, the evaluation of thermoregulation, circulatory problems, perfusion, thermoregulation issues, heat/cold stress and infections.

### SUMMARY

Accordingly, one or more embodiments provide a measuring system for non-invasive determination of one or more temperatures of a subject. The system comprises a body of engagement configured to engage with and/or support a subject, multiple coupling sensors, multiple temperature sensors, and one or more processors configured to execute computer program modules. The coupling sensors, in some embodiments, generate coupling signals conveying electrical and/or thermal coupling information with the subject. The coupling sensors may be carried by the body of engagement. The temperature sensors generate output signals conveying temperatures or a temperature map of the subject. The temperature sensors are carried by the body of engagement. The computer program modules comprise a coupling module and a temperature determination module. The coupling module is configured to determine coupling levels for individual ones of the temperature sensors based on the coupling signals generated by the coupling sensors. The temperature determination module is configured to determine multiple temperatures of the subject based on the output signals and, optionally, the determined coupling levels.

It is yet another aspect of one or more embodiments to provide a method of non-invasive determination of one or more temperatures of a subject. The method comprises engaging a subject with a body of engagement; generating coupling signals conveying electrical and/or thermal coupling information with the subject at or near a point of engagement between the subject and the body of engagement; generating output signals conveying temperatures of the subject at or near a point of engagement between the subject and the body of engagement; determining coupling levels for individual ones of multiple temperature sensors based on the coupling signals; and determining multiple temperatures of the subject based on the output signals and, optionally, the determined coupling levels.

It is yet another aspect of one or more embodiments to provide a system configured to provide non-invasive determination of one or more temperatures of a subject. The system comprises means for engaging a subject with a body; coupling means for generating coupling signals conveying electrical and/or thermal coupling information with the subject at or near a point of engagement between the subject and the means for engaging; temperature means for generating output signals conveying temperatures of the subject at or near a point of engagement between the subject and the means for engaging; means for determining coupling levels for the temperature means based on the coupling signals; and means for determining multiple temperatures of the subject based on the output signals and, optionally, the determined coupling levels.

These and other aspects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of any limits.

### BRIEF DESCRIPTION OF THE DRAWINGS

FIGS. 1A-1B-1C schematically illustrate a system for non-invasive determination of one or more temperatures of a subject, in accordance with one or more embodiments;

FIG. 2 schematically illustrates a measuring system in accordance with one or more embodiments;

FIG. 3 illustrates a graph of multiple temperatures measured over time in accordance with one or more embodiments;

FIGS. 4A-4B illustrate temperature maps in accordance with one or more embodiments; and

FIG. 5 illustrates a method for non-invasive determination of one or more temperatures of a subject, in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

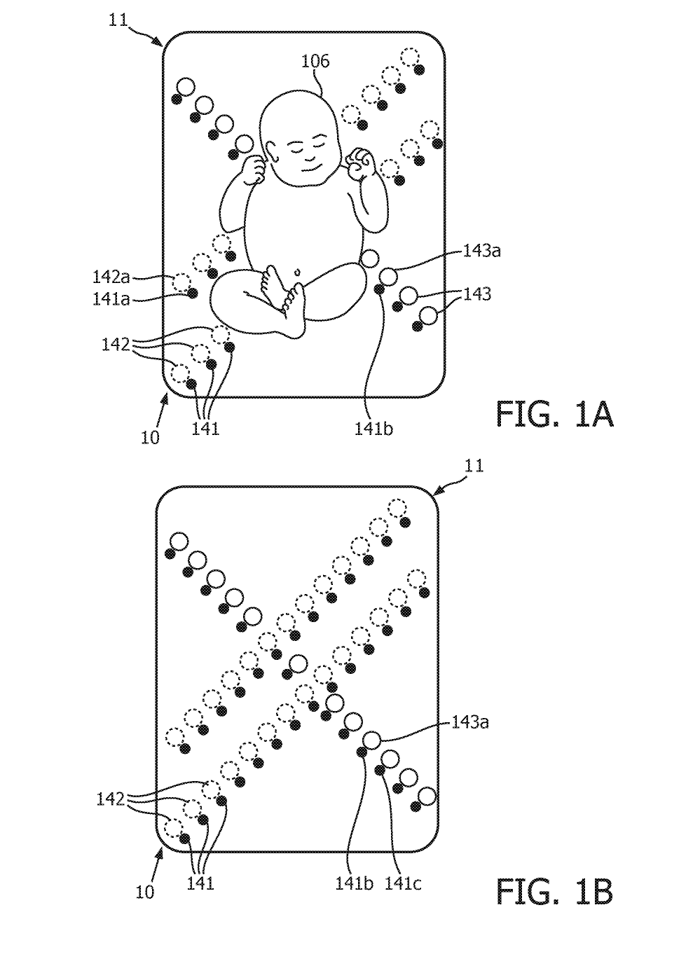

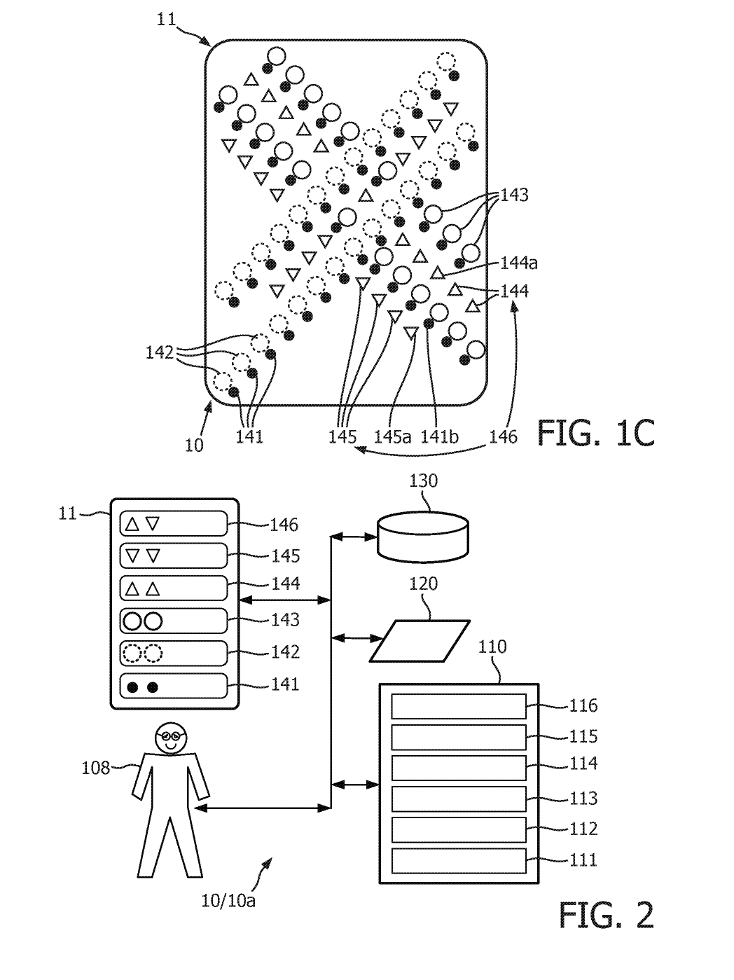

FIG. 1A illustrates (a top-view of) measuring system 10 for non-invasive determination of one or more temperatures of a subject 106. Measuring system 10 may interchangeably be referred to as system 10. System 10 may include one or more of a body of engagement 11, multiple coupling sensors 141, multiple temperature sensors 142, one or more zero-heat-flux temperature sensors 143, and/or other components (including components illustrated in other figures as being included in system 10). Body of engagement 11 may interchangeably be referred to as “structure of engagement,” “structure,” “support-structure of engagement,” or “support-structure.” By way of non-limiting example, FIG. 2 schematically illustrates system 10, which may further include one or more thermal adjustment elements 146 (e.g. one or more heating elements 144, e.g. an array of LEDs and/or one or more cooling elements 145, e.g. thermoelectric cooling elements), one or more processors 110, an electronic storage 130, a user interface 120, and/or other components and/or computer program modules. The computer program modules may include one or more of a coupling module 111, a temperature determination module 112, a map module 113, a tracking module 114, a target module 115, a control module 116, and/or other modules. Also illustrated in FIG. 2 is a user 108 of system 10 such as, by way of non-limiting example, a care-giver, a therapy-decision-maker, and/or a medical professional.

Non-invasive determination of one or more temperatures of a subject, in particular neonates and/or infants, may contribute to thermal protection and/or maintenance of recommended temperatures. Measuring temperatures of a subject may be important in many clinical situations, including but not limited to neonates in a neonatal intensive care unit (NICU). The multiple temperatures may include peripheral temperatures at various locations, core temperatures at or near different parts of the body, and/or other temperatures. For example, peripheral temperatures may include skin temperatures of hands, feet, and/or other body parts. For example, core temperatures may include (estimated, determined, measured, and/or otherwise approximated) temperatures of various organs and/or body parts, including but not limited to the brain, the heart, the abdomen, the chest, and/or other organs and/or body parts. As used herein, the term “non-invasive” may refer to the absence of adhesives to keep sensors in place and/or the absence of physical equipment penetrating or adhering to the skin or being inserted in any manner into the subject. Adhesive (temperature) sensors may damage the skin and cause stress and/or pain when used. Information regarding on or more temperatures of a subject (as well as information regarding changes over time in one or more such temperatures) may be medically and/or diagnostically relevant. For example, issues regarding thermoregulation, circulatory function, perfusion, infections, oxygen saturation, and/or other conditions of a subject may be diagnosed, monitored, treated, and/or otherwise benefit by virtue of having more and/or more accurate information regarding one or more temperatures of the subject. Medical conditions and/or issues mentioned in this disclosure are intended to be exemplary and without limitation.

Referring to FIG. 1A, body of engagement 11 is configured to engage with a subject 106, e.g. a neonate and/or infant. In some embodiments, body of engagement 11 may be implemented as a (subject) support structure configured to support subject 106 thereon. A subject support structure may be a mattress, a bed, a pad, a blanket, a wrap, a pillow, an incubator, and/or other structure suitable to engage and/or support a subject 106, e.g. a neonate and/or infant. In some embodiments, body of engagement 11 may be an article of clothing configured to be worn by and/or wrapped around subject 106. Body of engagement 11 may be configured to carry one or more sensors, e.g. one or more temperature sensors 142. As depicted in FIG. 1A, body of engagement 11 may be wrapped around subject 106 such that multiple coupling sensors 141 and multiple temperature sensors 142 engage, touch, and/or (electrically and/or thermally) couple with subject 106.

As used herein, a generic reference to a temperature sensor or a reference to multiple temperature sensors may use the term “temperature sensor(s) 142,” or variations thereof using the reference numeral “142,” whereas a specific individual temperature sensor may be referred to by appending a character to that reference numeral, e.g. “temperature sensor 142a”, depicted in FIG. 1A. Likewise, FIG. 1A depicts multiple coupling sensors 141 as well as specific coupling sensors referred to as coupling sensor 141a, coupling sensor 141b, and coupling sensor 141c, and multiple zero-heat-flux temperature sensors 143 as well as a specific zero-heat-flux temperature sensor 143a. Other temperature sensors, coupling sensors and zero-heat-flux temperature sensors are depicted in FIG. 1A, but not individually labeled with a reference number. As used in any of the figures, similar types of sensors may be depicted by similar schematic symbols. For example, temperature sensor(s) 142 are depicted using similar symbols in FIGS. 1A-1B-1C and FIG. 2. The disclosure is not limited to the number or position of any sensors depicted in any of the figures. As used herein, the term “measure” refers to any combination of measuring, estimating, and/or approximating based on output generated by one or more sensors. As used herein, the term “measurement” refers to any combination of one or more measurements, estimations, and/or approximations based on output generated by one or more sensors.

Temperature sensor(s) 142 may be configured to generate output signals conveying temperatures of a subject and/or output signals conveying information related in a predictable manner (e.g. through a mathematical relationship) to one or more temperatures of a subject. In some embodiments, temperature sensor(s) 142 may include one or more zero-heat-flux temperature sensors 143. Temperature sensor(s) may be supported and/or carried by body of engagement 11. Zero-heat-flux temperature sensor(s) 143 may be configured to create thermal insulation between two objects (e.g. body of engagement 11 and subject 106). Zero-heat-flux temperature sensor(s) 143 operate according to the thermal principle known as the zero-heat flux principle, which may be described, e.g., in one or more related applications incorporated by reference into the present application. In some embodiments, temperature sensor(s) 142 may be used to determine one or more peripheral temperatures of subject 106. In some embodiments, zero-heat-flux temperature sensor(s) 143 may be used to determine one or more core temperatures of subject 106. In some embodiments, one or more temperature sensors 142 may be configured to determine an ambient temperature around and/or near subject 106.

Coupling sensors 141 may be configured to generate signals (interchangeably referred to herein as output signals or coupling signals) conveying electrical, thermal, and/or other coupling information between two objects (e.g. the coupling sensor itself and subject 106). Coupling sensor(s) 141 may be supported and/or carried by body of engagement 11. In some embodiments, coupling sensor(s) may include one or more pressure sensors and/or one or more capacitive sensors. Signals and/or information conveyed by coupling sensor(s) 141 may be referred to as coupling information. One or more coupling sensors 141 may be associated with one or more temperature sensors, including but not limited using a 1-to-1 association (e.g. for co-located sensor pairs of a temperature sensor and a coupling sensor). By way of non-limiting example, referring to FIG. 1A, coupling sensors 141a, 141b, and 141c may be associated with different (zero-heat-flux) temperature sensors. In some embodiments, coupling information may be conveyed by the intensity, strength, magnitude, and/or level of the signal generated by coupling sensor(s) 141. For example, in some embodiments, an individual coupling sensor 141 may emit a signal (e.g. an electromagnetic signal) having known characteristics (including but not limited to a known frequency, shape, magnitude, and/or other characteristic of an electromagnetic signal). The coupling information for the individual coupling sensor 141 may be based on how well the emitted signal is received. In case of good and/or strong coupling between the coupling sensor and subject 106, the received signal may have a greater magnitude than compared to a poor and/or weak coupling between the coupling sensor and subject 106.

In some embodiments, an individual coupling sensor may be associated with multiple temperature sensors. In some embodiments, multiple coupling sensors may be associated with an individual temperature sensor. In some embodiments, association between one or more coupling sensors 141 and one or more temperature sensors 142 may be based on proximity (including but not limited to a weighted association of the information from a temperature sensor based on coupling information from the nearest multiple coupling sensors). In some embodiments, an individual temperature sensor and an individual coupling sensor may be integrated, embedded, and/or otherwise combined into a single unit, component, and/or device capable of the joint features and functionality attributed herein to an individual temperature sensor and an individual coupling sensor.

By way of non-limiting example, coupling sensor 141a depicted in FIG. 1A may be associated with temperature sensor 142a. For example, coupling information from coupling sensor 141a may be used to qualify information from temperature sensor 142a. Information from temperature sensor 142a may be deemed useful and/or reliable based on the information from coupling sensor 141a. For example, information from temperature sensor 142a may be discarded based on poor and/or weak coupling between coupling sensor 141a and subject 106, as may be conveyed through coupling information from coupling sensor 141a. The relative position of coupling sensor 141a in relation to temperature sensor 142a as depicted in FIG. 1A (near the lower section on the right-hand side of temperature sensor 142a) is merely exemplary and not intended to be limiting in any way.

The view of body of engagement 11 is partially obscured in FIG. 1A by subject 106. FIG. 1B depicts the same body of engagement 11 (and the same system 10) as depicted in FIG. 1A without subject 106 obscuring the view. Body of engagement 11 may include multiple temperature sensors 142 and multiple coupling sensors 141. The sensors depicted in FIG. 1B may be arranged to form a set, pattern, grid, and/or other predetermined shape. As depicted in FIG. 1, the sensors of system 10 may be arranged in multiple diagonal lines.

In some embodiments, system 10 includes one or more thermal adjustment elements 146 configured to adjust one or more temperatures of subject 106. Thermal adjustment elements 146 may include one or more heating elements 144 and/or one or more cooling elements 145. In some embodiments, an individual thermal adjustment element 146 may be configured to either heat or cool (at least a region and/or part of) subject 106. In some embodiments, one or more thermal adjustment elements 146 may be associated with one or more coupling sensors 141. For example, as depicted in FIG. 1C, coupling sensor 141b may be associated with cooling element 145a, e.g. based on proximity. In some embodiments, the same individual coupling sensor 141 may be associated with both a temperature sensor 142 and a thermal adjustment element 146. Resulting signals or information from any sensors may be transmitted to processor 110, user interface 120, electronic storage 130, and/or other components of system 10. This transmission may be wired and/or wireless.

By way of illustration, FIG. 1C illustrates another embodiment of the measuring system described in this disclosure, this embodiment depicted as system 10a that includes body of engagement 11a. System 10a of FIG. 1C may include substantially the same components and functionality as attributed to system 10 of FIG. 1B, except for the number, placement, and type of some of the sensors used. Additionally, as depicted in FIG. 1C, system 10a and body of engagement 11a may include one or more thermal elements 146, for example multiple heating elements 144 and multiple cooling elements 145. As used herein, a generic reference to a heating element or a reference to multiple heating elements may use the term “heating element(s) 144,” or variations thereof using the reference numeral “144,” whereas a specific individual heating element may be referred to by appending a character to that reference numeral, e.g. “heating element 144a”, depicted in FIG. 1C. Likewise, FIG. 1C depicts multiple cooling elements 145 as well as a specific cooling element referred to as cooling element 145a.

Referring to system 10 of FIG. 2 (and/or system 10a, as used interchangeably in reference to FIG. 2), system 10 may include electronic storage 130 comprising electronic storage media that electronically stores information. The electronic storage media of electronic storage 130 includes one or both of system storage that is provided integrally (i.e., substantially non-removable) with system 10 and/or removable storage that is connectable to system 10 via, for example, a port (e.g., a USB port, a FireWire port, etc.) or a drive (e.g., a disk drive, etc.). Electronic storage 130 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 130 stores software algorithms, information determined by processor 110, information received via user interface 120, and/or other information that enables system 10 to function properly. For example, electronic storage 130 may record or store (a set of) one or more temperatures and/or parameters derived from output signals measured (e.g. over time) by one or more sensors (as discussed elsewhere herein), and/or other information. Electronic storage 130 may be a separate component within system 10, or electronic storage 130 may be provided integrally with one or more other components of system 10 (e.g., processor 110).

Referring to FIG. 2, system 10 may include user interface 120 configured to provide an interface between system 10 and a user (e.g., user 108, a caregiver, a therapy decision-maker, etc.) through which the user can provide information to and receive information from system 10. This enables data, results, and/or instructions and any other communicable items, collectively referred to as “information,” to be communicated between the user and system 10. Examples of interface devices suitable for inclusion in user interface 120 include a keypad, buttons, switches, a keyboard, knobs, levers, a display screen, a touch screen, speakers, a microphone, an indicator light, an audible alarm, and a printer. Information may e.g. be provided to user 108 by user interface 120 in the form of auditory signals, visual signals, tactile signals, and/or other sensory signals.

By way of non-limiting example, in certain embodiments, user interface 120 includes a radiation source capable of emitting light. The radiation source includes one or more of an LED, a light bulb, a display screen, and/or other sources. User interface 120 may control the radiation source to emit light in a manner that conveys information to, e.g., user 108 related to, e.g., a breaching of a predetermined temperature threshold by subject 106.

It is to be understood that other communication techniques, either hard-wired or wireless, are also contemplated herein as user interface 120. For example, in one embodiment, user interface 120 is integrated with a removable storage interface provided by electronic storage 130. In this example, information is loaded into system 10 from removable storage (e.g., a smart card, a flash drive, a removable disk, etc.) that enables the user(s) to customize the implementation of system 10. Other exemplary input devices and techniques adapted for use with system 10 as user interface 120 include, but are not limited to, an RS-232 port, RF link, an IR link, modem (telephone, cable, Ethernet, internet or other). In short, any technique for communicating information with system 10 is contemplated as user interface 120.

Referring to FIG. 2, processor 110 is configured to provide information processing capabilities in system 10. As such, processor 110 includes one or more of a digital processor, an analog processor, a digital circuit designed to process information, an analog circuit designed to process information, and/or other mechanisms for electronically processing information. Although processor 110 is shown in FIG. 2 as a single entity, this is for illustrative purposes only. In some embodiments, processor 110 includes a plurality of processing units.

As is shown in FIG. 2, processor 110 is configured to execute one or more computer program modules. The one or more computer program modules include one or more of a coupling module 111, a temperature determination module 112, a map module 113, a tracking module 114, a target module 115, a control module 116, and/or other modules. Processor 110 may be configured to execute modules 111-116 by software; hardware; firmware; some combination of software, hardware, and/or firmware; and/or other mechanisms for configuring processing capabilities on processor 110.

It should be appreciated that although modules 111-116 are illustrated in FIG. 2 as being co-located within a single processing unit, in implementations in which processor 110 includes multiple processing units, one or more of modules 111-116 may be located remotely from the other modules. The description of the functionality provided by the different modules 111-116 described below is for illustrative purposes, and is not intended to be limiting, as any of modules 111-116 may provide more or less functionality than is described. For example, one or more of modules 111-116 may be eliminated, and some or all of its functionality may be provided by other ones of modules 111-116. Note that processor 110 may be configured to execute one or more additional modules that may perform some or all of the functionality attributed below to one of modules 111-116.

Sensors in this disclosure may be configured to generate output signals in an ongoing manner, e.g. throughout the day. This may include generating signals intermittently, periodically (e.g. at a sampling rate), continuously, continually, at varying intervals, and/or in other ways that are ongoing during at least a portion of period of a day, week, month, or other duration. The sampling rate may be about 0.001 second, 0.01 second, 0.1 second, 1 second, about 10 seconds, about 1 minute, and/or other sampling rates. It is noted that multiple individual sensors may operate using different sampling rates, as appropriate for the particular output signals and/or (frequencies related to particular) parameters derived therefrom. For example, in some embodiments, the generated output signals may be considered as a vector of output signals, such that a vector includes multiple samples of information conveyed related to one or more temperatures of subject 106. Different temperatures may be related to different vectors. A particular temperature determined in an ongoing manner from a vector of output signals may be considered as a vector of that particular temperature.

Coupling module 111 of system 10 in FIG. 2 is configured to determine coupling levels for one or more sensors of system 10, including but not limited to one or more coupling sensors 141, one or more temperature sensors 142, one or more zero-heat-flux temperature sensors 143, and/or other sensors. As used herein, the term “coupling level” may refer to coupling strength (e.g. of electrical signals), and/or signal strength (e.g. of electrical signals). In some embodiments, coupling levels may be based on pressure levels, capacitive levels, and/or other types of levels and/or combinations thereof that may indicate whether (and/or to what extent) the output signal from a sensor should be deemed reliable. Alternatively, and/or simultaneously, in some embodiments, a coupling level may indicate whether the output signal from a sensor should be discarded, e.g. in favor of stronger and/or more reliable signals from other sensors.

In some embodiments, coupling module 111 may be configured to determine individual coupling levels for individual temperature sensors 142. In some embodiments, determinations by coupling module 111 may be based on one or more coupling signals generated by coupling sensors 141. For example, a coupling level for temperature sensor 142a may be based on coupling information from coupling sensor 141a. In some embodiments, individual temperature sensors 142 may be associated with individual coupling sensors 141, and/or vice versa. In some embodiments, information from an individual temperature sensor 142 may be weighted according to the coupling levels of multiple nearby coupling sensors 141. The coupling level for an individual temperature sensor 142 may change over time, for example between measurements taken of individual coupling sensors 141. Changes in coupling levels over time may, for example, be caused by movement of subject 106. Coupling levels from coupling sensors 141 may be ordered, ranked, and/or otherwise compared to coupling levels from one or more other coupling sensors. For example, coupling levels from coupling sensors 141 within a predetermined distance of each other and/or another sensor may be compared with each other and/or with one or more thresholds. Coupling levels from coupling sensors 141 may be compared based on the output signals being generated within the same period, duration, and/or window. By way of non-limiting example, in some embodiments coupling sensors 141 may be configured to generate output signals at a sampling rate of 1 second per measurement. Coupling module 111 may be configured to determine coupling levels for some or all coupling sensors 141 at the same or similar sampling rate such that changing coupling levels may be reevaluated at the same or similar sampling rate to determine whether to use or discard corresponding temperature measurements from associated temperature sensors 142.

Temperature determination module 112 of system 10 in FIG. 2 is configured to determine one or more temperatures of subject 106. The temperatures may include one or more peripheral temperatures at various locations, one or more core temperatures at or near different parts of the body, and/or other temperatures. In some embodiments, temperature determination module 112 may be configured to determine multiple temperatures and/or multiple types of temperatures of subject 106, including but not limited to one or more peripheral temperatures and/or one or more core temperatures. Determinations by temperature determination module 112 may be based on one or more output signals from one or more temperature sensors 142, one or more coupling signals from one or more coupling sensors 141, and/or one or more coupling levels determined by coupling module 111, one or more determinations by map module 113, and/or any combination thereof. For example, output signals from temperature sensors that correspond to a low coupling level (e.g. compared to a coupling level threshold and/or to coupling levels of other sensors) may be discarded, for example in favor of output signals from other temperature sensors that correspond to a high or higher coupling level (e.g. compared to the same or a different coupling level threshold and/or to coupling levels of other sensors).

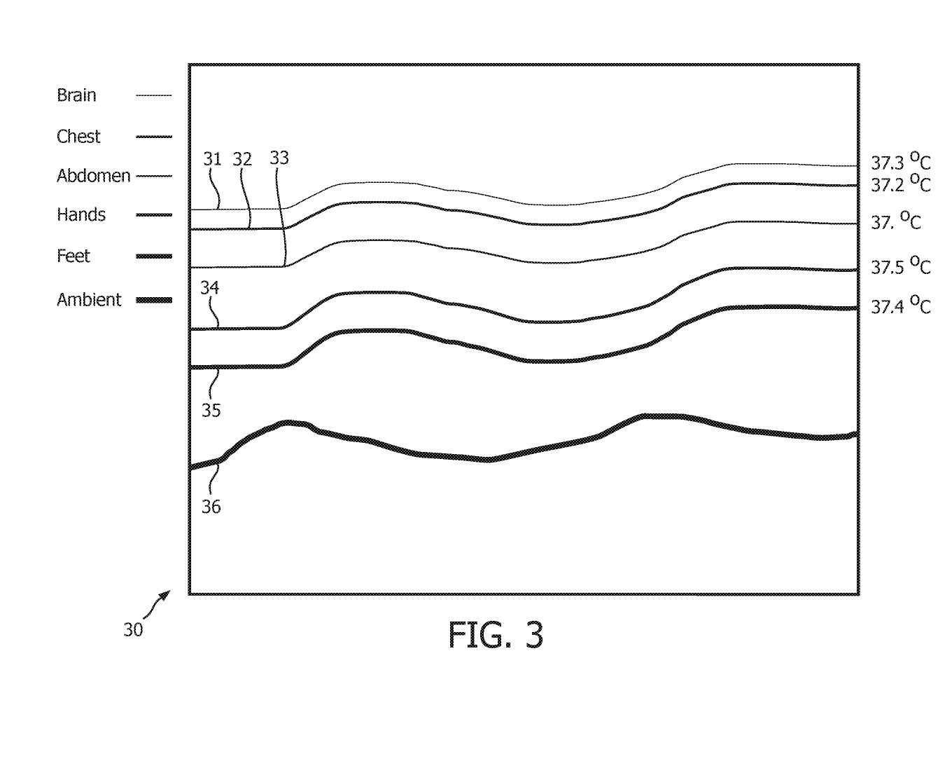

In some embodiments, temperature sensors 142 may include one or more zero-heat-flux temperature sensors 143. Temperature determination module 112 may be configured to determine one or more core temperatures of subject 106 based on output signals generated by zero-heat-flux temperature sensors 143. Alternatively, and/or simultaneously, one or more determined core temperatures of subject 106 may further be based on one or more coupling levels determined by coupling module 111. For example, a particular core temperature may be based on a coupling level for zero-heat-flux temperature sensor 143a, which may be based on coupling information from coupling sensor 141b. Temperature determination module 112 may be configured to determine multiple temperatures of subject 106 over time. By way of non-limiting example, FIG. 3 illustrates a graph 30 including multiple temperatures (in ° C.) measured over time (along the X-axis), including temperatures for the brain (31), chest (32), abdomen (33), hands (34), feet (35), and ambient temperature (36). By way of non-limiting example, brain temperature 31 may be a core temperature and feet temperature 35 may be a peripheral temperature.

In some embodiments, temperature determination module 112 may be configured to determine one or more temperatures of subject 106 without using or needing coupling information. For example, determinations by temperature determination module 112 may be based on one or more of positional information (described elsewhere herein), and/or a temperature map of subject 106 (e.g. determined by map module 113).

In some embodiments, system 10 may include one or more sensors configured to generate output signals conveying positional information of subject 106. Positional information of subject 106 may include information about the relative position of subject 106 (and/or one or more body parts of subject 106) as compared to one or more of system 10, body of engagement 11, a support structure in which subject 106 has been placed, an incubator, a crib, all or part of a NICU, and/or another object. In some embodiments, positional information may be derived from and/or based on coupling information. In some embodiments, positional information may be derived from (e.g. deduced from) one or more temporal variations of one or more temperatures and/or variations of the temperature map of subject 106 over time, for example in conjunction with a (parameterized) model that does not use coupling information. Alternatively, and/or simultaneously, in some embodiments, positional information may be derived from and/or based on information conveyed by one or more image sensors. For example, positional information may be based on information from a (video and/or photography) camera. In some embodiments, positional information may be determined by coupling module 111. Alternatively, and/or simultaneously, in some embodiments, positional information may be derived from and/or based on information conveyed by one or more temperature sensors, e.g. in combination with coupling information. For example, positional information may be based on (e.g. derived, deduced, and/or inferred from) a temperature map of a subject, e.g. as determined by map module 113.

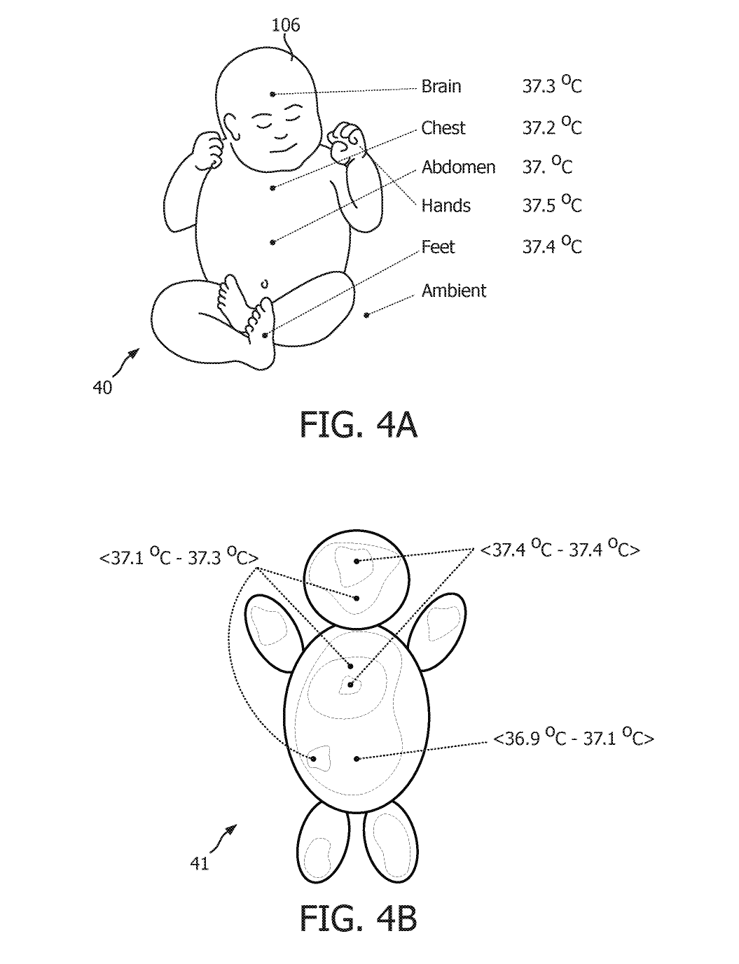

Map module 113 of system 10 in FIG. 2 is configured to determine and/or construct a temperature map of subject 106 based on temperatures determined by temperature determination module 112 and/or positional information of subject 106. As used herein, the term “temperature map” may be used interchangeably with the terms “temperature profile” and “graphical temperature representation”. For example, a temperature map may depict an image subject 106 combined with information about different relevant temperatures. By way of non-limiting illustration, FIG. 4A illustrates a temperature map 40 of subject 106. In some embodiments, the image used in temperature map 40 may be an actual representation (e.g. a photograph) of subject 106. In some embodiments, the image used in temperature map 40 may be a real-time representation (e.g. a video image) of subject 106. Temperature map 40 may, by way of non-limiting example, include the same or similar temperatures as depicted in FIG. 3, including temperatures for the brain, chest, abdomen, hands, feet, and ambient temperature. By way of example, the end temperatures (i.e. the right-most temperatures depicted) from graph 30 (FIG. 3) are depicted as the current temperatures in temperature map 40 in FIG. 4A. Temperature map 40 may be 2-dimensional or more-then-2-dimensional, for example 3-dimensional.

In some embodiments, a temperature map of subject 106 may be based on a (parameterized) model using multiple determined temperatures of subject 106. Optionally, the model may use coupling information. Optionally, the model may use positional information of subject 106, e.g. for embodiments in which positional information is determined independently of a temperature map. In some embodiments, a temperature map may be inferred from multiple determined temperatures of subject 106 and positional information of subject 106.

In some embodiments, a temperature map may depict regions of subject 106 having the same or similar temperature, such as a heat map. Such regions may for example be indicated using different colors. In some embodiments, the image used in temperature map 41 may be an actual representation (e.g. a photograph) of subject 106, or a schematic representation (including head, torso, arms, and legs) as depicted in FIG. 4B. This list of body parts is exemplary and not intended to be limiting in any way. By way of non-limiting example, FIG. 4B illustrates a temperature map 41 that depicts regions of subject 106 having similar temperatures. For example, two regions are indicated as having a temperature between 37.3° C. and 37.4° C., three regions are indicated as having a temperature between 37.1° C. and 37.3° C., one region is indicated as having a temperature between 36.9° C. and 37.1° C. The different temperatures (or temperature ranges) may be indicated in a temperature map using different colors. In some embodiments, the image used in temperature map 41 to represent subject 106 may be a real-time 3-dimensional representation of subject 106. By way of non-limiting example, one or both of the two described regions may be core temperatures and one or more of the temperatures and/or regions associated with the extremities of subject 106 may be peripheral temperatures.

Tracking module 114 of system 10 in FIG. 2 is configured to track changes in one or more temperatures over time. Tracking module 114 may be configured to track changes in the span of about 10 minutes, about an hour, about 2 hours, about 4 hours, about 8 hours, about 12 hours, about 24 hours, about 48 hours, about 72 hours, about a week, about a month, about 2 months, and/or other amounts of time. Relatively slow changes in temperature (compared to the sampling rate) may indicate a change in a medical condition that might be noteworthy. For example, a particular temperature (e.g. determined by temperature determination module 112) may rise or fall outside an acceptable and/or preferred range for such a temperature. In some embodiments, tracking module 114 may be configured to determine whether a difference between two temperatures increases or decreases over time, and/or whether such a change falls outside an acceptable and/or preferred range for such a difference. For example, tracking module 114 may be configured to determine whether the peripheral temperature of one or both hands differs more than a predetermined maximum difference threshold from the temperature of the brain. For example, tracking module 114 may be configured to determine whether the peripheral temperatures of the extremities are more than a predetermined maximum difference threshold apart from each other.

In some embodiments, tracking module 114 may be configured to determine whether one or more temperatures and/or changes in temperatures indicate significant information pertinent to diagnostic purposes, as described elsewhere herein. System 10 may be configured to measure other patient-specific parameters as needed to support the process of such determinations, including but not limited to physiological parameters, respiratory parameters, and/or any other medically relevant parameters and/or combinations thereof. For example, a particular predetermined combination of a change in heart rate, a change in respiratory rate, and a change in one or more temperatures may indicate a particular medical condition or emergency that may be noteworthy to a user and/or caregiver. As used herein, the term “predetermined” may refer to a determination that has been made prior to usage of system 10 on a particular subject. For example, a programmed relation, value, or threshold may be referred to as predetermined In some embodiments, tracking module 114 may be configured to notify and/or alert a user or caregiver responsive to one or more determinations (described in this disclosure) having been made.

Target module 115 is configured to obtain and/or determine one or more target temperatures and/or target temperature ranges for subject 106. For example, the one or more target temperatures may be specific to the type (e.g. core, peripheral, or other) and/or location of the measurements (e.g. which body part, organ, area, and/or region of subject 106). One or more target temperatures and/or target temperature ranges may be recommended by one or more medical professionals as being desirable for subject 106. Determined temperatures (e.g. by temperature determination module 112) may be compared to one or more target temperatures and/or target temperature ranges. For example, a target temperature range for the brain temperature may be between 37.2° C. and 37.5° C. Responsive to a determination that a brain temperature falls outside of the corresponding target temperature range, system 10 may be configured to (attempt to) adjust the relevant temperature of subject 106, as described elsewhere herein.

Control module 116 of system 10 in FIG. 2 is configured to control one or more thermal adjustment elements 146. In some embodiments, control module 116 may be configured to control one or more thermal adjustment elements 146 in accordance with a therapy regimen. In some embodiments, control module 116 may be configured to control one or more thermal adjustment elements 146 to adjust one or more of the determined temperatures (e.g. as determined by temperature determination module 112). In some embodiments, control module 116 may be configured to control one or more thermal adjustment elements 146 based on one or more comparisons between a determined temperature and a target temperature (and/or target temperature range). In some embodiments, control module 116 may be configured to control one or more thermal adjustment elements 146 in accordance with one or more determined target temperatures and/or target temperature ranges (e.g. as determined by target module 115). For example, responsive to a comparison between a target temperature and a corresponding determined temperature, control module 116 may be configured to increase or decrease a particular body part, organ, area, and/or region of subject 106. This may be referred to as heating or cooling, respectively. For example, heating may be accomplished using one or more heating elements 144; cooling may be accomplished using one or more cooling elements 145. Selection of one or more particular thermal adjustment elements 146 may depend on locality and/or proximity of the corresponding temperature sensor(s) 142. Alternatively, and/or simultaneously, in some embodiments, selection of one or more particular thermal adjustment elements 146 may depend on coupling levels as determined for nearby coupling sensors 141, by virtue of the notion that weak electrical and/or thermal coupling may affect the efficacy of a thermal adjustment element at the same or similar location (e.g. for a thermal adjustment element 146 located close to a coupling sensor 141).

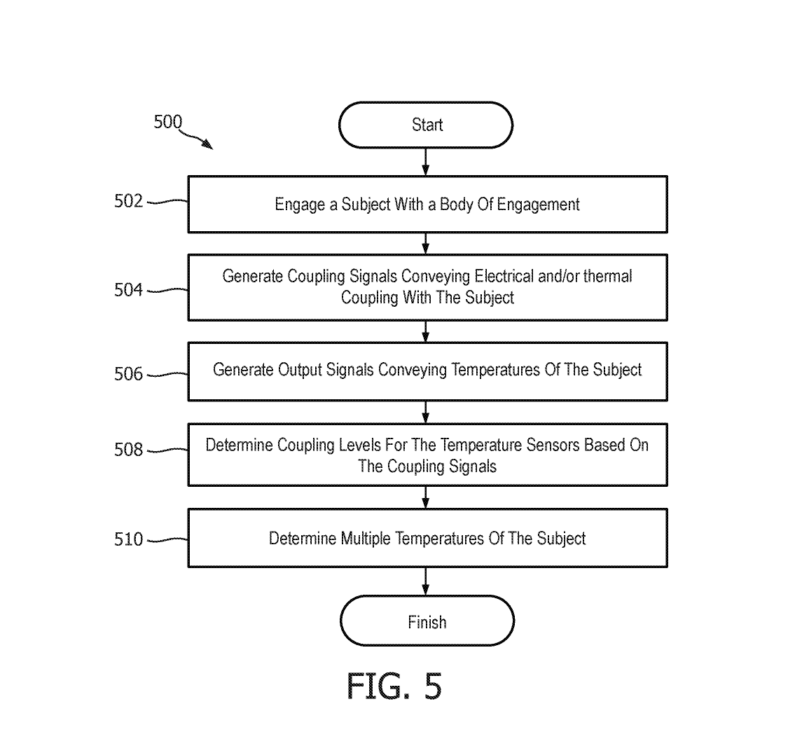

FIG. 5 illustrates a method 500 to determine one or more temperatures of a subject. The operations of method 500 presented below are intended to be illustrative. In certain embodiments, method 500 may be accomplished with one or more additional operations not described, and/or without one or more of the operations discussed. Additionally, the order in which the operations of method 500 are illustrated in FIG. 5 and described below is not intended to be limiting.

In certain embodiments, method 500 may be implemented in one or more processing devices (e.g., a digital processor, an analog processor, a digital circuit designed to process information, an analog circuit designed to process information, and/or other mechanisms for electronically processing information). The one or more processing devices may include one or more devices executing some or all of the operations of method 500 in response to instructions stored electronically on an electronic storage medium. The one or more processing devices may include one or more devices configured through hardware, firmware, and/or software to be specifically designed for execution of one or more of the operations of method 500.

At an operation 502, a subject engages with a body of engagement. In some embodiments, operation 502 is performed by a body of engagement the same as or similar to body of engagement 11 (shown in FIG. 1A and described herein).

At an operation 504, coupling signals are generated conveying electrical and/or thermal coupling with the subject at or near a point of engagement between the subject and the body of engagement. In some embodiments, operation 504 is performed by coupling sensors the same as or similar to coupling sensors 141 (shown in FIG. 1A and described herein).

At an operation 506, output signals are generated conveying temperatures of the subject at or near a point of engagement between the subject and the body of engagement. In some embodiments, operation 506 is performed by temperature sensors the same as or similar to temperature sensors 142 (shown in FIG. 1A and described herein).

At an operation 508, coupling levels are determined for individual ones of the temperature sensors based on the coupling signals. In some embodiments, operation 508 is performed by a coupling module the same as or similar to coupling module 111 (shown in FIG. 2 and described herein).

At an operation 510, multiple temperatures of the subject are determined based on the output signals and the determined coupling levels. In some embodiments, operation 510 is performed by a temperature determination module the same as or similar to temperature determination module 112 (shown in FIG. 2 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although this description includes details for the purpose of illustration based on what is currently considered to be the most practical and preferred embodiments, it is to be understood that such detail is solely for that purpose and that the disclosure is not limited to the disclosed embodiments, but, on the contrary, is intended to cover modifications and equivalent arrangements that are within the spirit and scope of the appended claims. For example, it is to be understood that, to the extent possible, one or more features of any embodiment are contemplated to be combined with one or more features of any other embodiment.

1. A measuring system for non-invasive determination and control of one or more temperatures of a subject, the system comprising:
a body of engagement configured to engage with and/or support a subject wherein the body of engagement is further configured to be wrapped around the subject;
multiple coupling sensors that generate coupling signals conveying electrical and/or thermal coupling information with the subject, wherein the coupling sensors are carried by the body of engagement;
multiple temperature sensors that generate output signals conveying temperatures of the subject, wherein the temperature sensors are carried by the body of engagement; and
one or more processors configured to execute computer program modules, the computer program modules comprising:
a coupling module configured to determine coupling levels for individual ones of the temperature sensors based on the coupling signals generated by the coupling sensors;
a temperature determination module configured to determine multiple temperatures of the subject based on the output signals and the determined coupling levels.

2. The system of claim 1, wherein the temperature sensors include a zero-heat-flux temperature sensor that generates output signals conveying a second temperature of the subject, wherein the zero-heat-flux temperature sensor is configured to create thermal insulation between the body of engagement and the subject, wherein the temperature determination module is configured such that the determined temperatures include a core temperature determined based on the output signals generated by the zero-heat-flux temperature.

3. The system of claim 1, wherein the coupling module is further configured to determine positional information of the subject in relation to the body of engagement based on the coupling signals generated by individual ones of the coupling sensors, the computer program modules further comprising:
a map module configured to construct a temperature map of the subject based on the determined temperatures and the determined positional information, wherein the determined temperatures include at least one extremity temperature of an extremity of the subject.

4. The system of claim 1, wherein the determined temperatures include a first temperature and a second temperature, wherein the first temperature is related to an extremity of the subject, and wherein the computer program modules further comprise:
a tracking module configured to determine a difference between the first temperature and the second temperature, wherein the tracking module is further configured to track whether the difference exceeds a predetermined threshold.

5. The system of claim 1, further comprising:
one or more thermal adjustment elements configured to adjust one or more determined temperatures of the subject,
wherein the computer program modules further comprise:
a target module configured to determine one or more target temperatures for one or more determined temperatures; and
a control module configured to control the one or more thermal adjustment elements in accordance with one or more determined target temperatures.

6. A method of non-invasive determination and control of one or more temperatures of a subject, the method comprising;
engaging a subject with a body of engagement by wrapping the body of engagement around the subject;
generating, by coupling sensors, coupling signals conveying electrical and/or thermal coupling information with the subject at or near a point of engagement between the subject and the body of engagement;
generating, by temperature sensors, output signals conveying temperatures of the subject at or near a point of engagement between the subject and the body of engagement;
determining coupling levels for individual ones of the temperature sensors based on the coupling signals; and
determining multiple temperatures of the subject based on the output signals and the determined coupling levels.

7. The method of claim 6, wherein the temperature sensors include a zero-heat-flux temperature sensor, wherein generating output signals conveying temperatures of the subject includes:
creating, by the zero-heat-flux temperature sensor, thermal insulation between the body of engagement and the subject, and
generating, by the zero-heat-flux temperature sensor, output signals conveying a second temperature of the subject,
wherein determining multiple temperatures includes determining a core temperature based on the output signals generated by the zero-heat-flux temperature sensor.

8. The method of claim 6, further comprising:
determining positional information of the subject in relation to the body of engagement based on the coupling signals;
constructing a temperature map of the subject based on the determined temperatures and the determined positional information, wherein the determined temperatures include at least one extremity temperature of an extremity of the subject.

9. The method of claim 6, wherein the determined temperatures include a first temperature and a second temperature, wherein the first temperature is related to an extremity of the subject, and further comprising:
determining a difference between the first temperature and the second temperature; and
tracking whether the difference exceeds a predetermined threshold.

10. The method of claim 6, further comprising:
determining one or more target temperatures for one or more determined temperatures;
controlling one or more thermal adjustment elements in accordance with one or more determined target temperatures, wherein controlling the one or more thermal adjustment elements is based on the determined temperatures.

11. A system configured to provide non-invasive determination and control of one or more temperatures of a subject, the system comprising;
means for engaging a subject with a body by wrapping the means for engaging around the subject;
coupling means for generating coupling signals conveying electrical and/or thermal coupling information with the subject at or near a point of engagement between the subject and the means for engaging;
temperature means for generating output signals conveying temperatures of the subject at or near a point of engagement between the subject and the means for engaging;
means for determining coupling levels for the temperature means based on the coupling signals; and
means for determining multiple temperatures of the subject based on the output signals and the determined coupling levels.

12. The system of claim 11, wherein the temperature means includes:
means for creating thermal insulation between the body of engagement and the subject, and
means for generating output signals conveying a second temperature of the subject,
wherein the means for determining multiple temperatures is configured to determine a core temperature based on the second temperature.

13. The system of claim 11, wherein the coupling means is further configured to determine positional information of the subject in relation to the body of engagement based on the coupling signals, the system further comprising:
means for constructing a temperature map of the subject, wherein operation of the means for constructing the temperature map is based on the determined temperatures and the positional information, and wherein the determined temperatures include at least one extremity temperature of an extremity of the subject.

14. The system of claim 11, wherein the determined temperatures include a first temperature and a second temperature, wherein the first temperature is related to an extremity of the subject, the system further comprising:
means for determining a difference between the first temperature and the second temperature; and
means for tracking whether the difference exceeds a predetermined threshold.

15. The system of claim 11, further comprising:
adjustment means for adjusting one or more determined temperature of the subject;
means for determining one or more target temperatures for one or more determined temperatures;
means for controlling the adjustment means in accordance with one or more determined target temperatures, wherein operation of the means for controlling is based on the determined temperatures.
